(19) United States
(12) Patent Application Publication
(10) Pub. No.: US 2005/0021151 A1
(43) Pub. Date: Jan. 27, 2005

(54) Bone implant

(76) Inventor: Klaus Landis, Heroldsberg (DE)

Correspondence Address: GIFFORD, KRASS, GROH, SPRINKLE;ANDERSON & CITKOWSKI, PC, 280 N OLD WOODARD AVE, SUITE 400, BIRMINGHAM, MI, 48009, US

(21) Appl. No.: 10/479,066

(22) PCT Filed: May 7, 2002
(86) PCT No.: PCT/EP02/05032

(30) Foreign Application Priority Data
May 29, 2001 (DE) 10126085.7

(51) Int. Cl. A61F 2/28

(52) U.S. Cl. 623/23.63; 623/23.72

(57) ABSTRACT

A bone implant made form a spongiform and/or cortical bone material of human or animal origin, comprises at least one surface region with several longitudinal adjacent drillings which do not extend through the bone implant.

The present invention relates to a bone implant, in particular to a bone implant made of a spongious and/or cortical bone material of human or animal origin.

Bone implants of the aforesaid kind are generally known. Said implants gained from human or animal bones are characterized in that, when implanted into the human body, they permit a good ongrowth of the tissue into which they are implanted, on the one hand, and are slowly and completely degraded by the body into which they have been implanted with a simultaneous build-up of the body's own bone tissue, on the other hand. The region in which the implant and the body tissue are in contact with one another, i.e. the ongrowth and rebuilding zone, however, generally forms a weak point in comparison with the bone material of the implant and also with that of the human body from a mechanical consideration.

A textured, demineralized and uniform section of a mammal bone is described in U.S. Pat. No. 5,112,354 which serves as a stiff collagen scaffold with small apertures for allogenic skeleton reconstruction. The allograft is manufactured by treatment of a section of a bone for the removal of soft tissue, by texturing of the bone surface for the provision of a specimen of bores of pre-determined size, density and depth and by subsequent demineralization of the bone section such that a stiff, insoluble collagen scaffold remains, which is suitable for osteoinduction after implanting. All these steps are carried out under minimal denaturalizing of the extra-cellular matrix proteins which remain bound to the collagen scaffold and which are necessary for the termination of the process of new bone building.

It is the object of the present invention to provide a bone implant of the aforementioned type which results in a better ongrowth and rebuilding zone with improved mechanical properties after implanting into the human body.

The object is satisfied by a bone implant having the features of claim 1.

A bone implant in accordance with the invention is made from spongious and/or from cortical bone material of human or animal origin, with, when different bone materials are used, these being able to be formed in the manner of layers or in the form of laminated material, e.g. with a spongious matrix and embedded reinforcing cortical inclusions.

At least one region of the surface of a bone implant in accordance with the invention has a plurality of elongate bores arranged adjacently and not passing through the bone implant. In this process, bores are understood to be blind holes manufactured mechanically, e.g. by drilling or milling, whose cross-section does not necessarily have to be circular. This surface region provided with bores forms an enlarged contact area contacted by the body's own tissue after implantation, which results in an improved healing of tissue contacting it and in an accelerated rebuilding of the bone implant in comparison to the use of a compact implant without bores at its surface. Furthermore, after the implantation, on the ingrowth of the body's own tissue into the bores, a mutually interleaved structure is formed of the bone implant with its bores and the walls bounding them and the body's own tissue growing into said bores in a finger-like manner. Whereas the material between the bores also has a high mechanical strength perpendicular and parallel to the direction of the bores in the initial phase of the degradation, substantial forces can likewise already be absorbed by the tissue parts grown into the bores, which results overall in improved mechanical properties in the ongrowth and rebuilding zone.

Furthermore, the bores can be used during the implanting for the purpose of gripping, moving and then positioning the implant using correspondingly formed holding and/or positioning tools. It is thereby possible to insert the implant into an opening of a bone with only minimum clearance.

The surface region with bores preferably extends up to an edge of the bone implant, with the bores in the edge region being outwardly open and thereby forming a ribbed section. This ribbing, which is lateral with respect to the surface region provided with bores, also permits a good lateral ingrowth of the bone implant and moreover permits the implant to be gripped, e.g. with forceps, very reliably in the region of the ribbing such that it can also be reliably manipulated even with only a low application of force. This ribbed surface can be achieved in that, after the introduction of the bores, the bone implant is cut in the surface region bearing the bores in a direction oblique or perpendicular to said surface region.

Advantageous embodiments of the invention are described in the description, in the Figures and in the dependent claims.

The shape and size of a bone implant in accordance with the invention are based on the application purpose and depend on the dimensions of the region into which it should be inserted. It can in particular be formed in pin shape, plate shape or also disk shape. Arched structures are naturally also possible. The height, width and/or length of the implant are preferably in the range of approximately 2 mm or larger.

The bores are preferably arranged in matrix-like form, i.e. in a regular pattern, preferably in a right angled pattern. Such an arrangement of the bores does not only permit a simpler manufacture of the bone implant in accordance with the invention, but also permits a better distribution of forces which act on the surface region provided with bores in the implanted state as well as a generally higher density of bores due to the uniform distribution of the bores.

The spacing of adjacent bores is preferably not larger than twice their maximum width. The spacing of two bores is understood in this process as the minimal distance between two wall regions of adjacent bores. A comparatively high density of the bores and thus of the surface available for the ingrowth and for the associated or following degradation is hereby achieved. Although it is generally possible for bores to border one another directly, so that an arrangement of columns not abutting one another and separated by the bores is possible, the spacing of the bores is particularly preferably larger than 0.01 mm in order to achieve a sufficient mechanical strength. The spacing of the bores particularly preferably lies between 0.01 mm and the maximum width of the bores.

The surface region can in this process preferably have between approximately 20 and 200 bores per cm².

The maximum width of the bores preferably lies between approximately 0.05 mm and approximately 2 mm, which permits a simple manufacture, on the one hand, but a higher density of bores with respect to the surface of the bone implant, on the other hand. Moreover, close bores are filled up faster by ingrowing bone tissue such that a carrying structure of the body's own tissue is formed faster which is interleaved with the implant, whereby a high strength of the ongrowth and rebuilding zone is achieved sooner. It is in particular also possible for the surface region to have bores of a different maximum value, with the different maximum widths being selected such that a particularly high density of bores is achieved relative to the surface of the bone implant provided with bores and/or a particularly high strength of this region is achieved.

The bores are elongate in accordance with the invention, which means that the depth of a bore is larger than its maximum width. In this process, a depth of the bore is particularly preferred which substantially surpasses, i.e. in particular by three times, the maximum width of the bore, since a particularly good engaging of the bone implant and of the ingrown tissue into one another and thus a particularly high mechanical strength of the ongrowth and rebuilding zone is thereby achieved. The depth of the bore can particularly preferably be between approximately 0.1 mm and approximately 10 mm, provided that the bores are elongate. The depth of the bores, however, does not necessarily have to be equal for all bores of a surface region.

Although the longitudinal axes of the bores do not necessarily have to be aligned parallel, it is preferred for the axes of the bores to be aligned substantially parallel. This does not only permit a simpler manufacture, but also a substantially higher density of bores as well as a more uniform load distribution. Moreover, holding and positioning tools can also be introduced more easily into such bores. The bores are preferably aligned perpendicular to the surface of the bone implant in the surface region bearing the bores such that the walls between the bores can easily absorb forces acting on the surface. If a different load direction is to be expected depending on the implant, the bores can naturally also be aligned diagonally with respect to the surface.

Although the surface region of the bone implant, which includes the bores, can generally consist of spongious material, it is preferably formed by compact bone material, i.e. for the bone material between the bores to be compact. A particularly high strength of the walls bearing the mechanical loads is hereby achieved.

The surface region with bores preferably extends up to an edge of the bone implant, with the bores in the edge region being outwardly open and thereby forming a ribbed section. This ribbing, which is lateral with respect to the surface region provided with bores, also permits a good lateral ingrowth of the bone implant and moreover permits the implant to be gripped, e.g. with forceps, very reliably in the region of the ribbing such that it can also be reliably manipulated even with only a low application of force. This ribbed surface can be achieved in that, after the introduction of the bores, the bone implant is cut in the surface region bearing the bores in a direction oblique or perpendicular to said surface region.

The bone implant in accordance with the invention can preferably have at least one further surface region in which further bores are introduced which each at least partly intersect at least one of the bores in the surface region. The further surface region can generally also be the surface region with bores; however, a further surface region is particularly preferred which is inclined toward the surface region. Furthermore, the bores do not necessarily have to pass through over their whole cross-section; it is generally rather sufficient for the bores to be connected in the intersection zone. In this further development, the bores, which optionally only partly intersect, form angled passages which bring about a particularly good connection between the implant and the ingrowing tissue on the ingrowth of tissue. A good absorption of tensile forces which act on the implant in the direction of the bores in particular also results. The implant is not

The bone implant in accordance with the invention can preferably have at least one further surface region in which further bores are introduced which each at least partly intersect at least one of the bores in the surface region. The further surface region can generally also be the surface region with bores; however, a further surface region is particularly preferred which is inclined toward the surface region. Furthermore, the bores do not necessarily have to pass through over their whole cross-section; it is generally rather sufficient for the bores to be connected in the intersection zone. In this further development, the bores, which optionally only partly intersect, form angled passages which bring about a particularly good connection between the implant and the ingrowing tissue on the ingrowth of tissue. A good absorption of tensile forces which act on the implant in the direction of the bores in particular also results.

The material of the bone implant preferably consists of preserved and sterile bone material. The bone implant is thereby capable of being stored and can be implanted without the risk of infection.

The bone implant is preferably of animal origin due to the better availability. Bovine bone material is particularly preferred due to the high strength of bovine bone.

The manufacture of an implant in accordance with the invention can start from a piece of a suitable bone material of sufficient size which is then further processed in a plurality of steps. The establishing of the bores can generally take place before, between or after the steps. In accordance with a method, native bone material can first be cleaned, for example in a saline solution, and then degreased in a known method*. After a deactivation and denaturing step, the material is preserved.
Translator's note: “in a” [known method] added by translator; evidently missing in the original.

The preservation of the bone material can take place, for example, by freeze-drying. However, preservation is preferably carried out by solvent dehydration of native bone material by means of an organic solvent miscible with water, e.g. methanol, ethanol, propanol, isopropanol, acetone, methyl ethyl ketone or mixtures of these solvents.

The blank produced in this manner is then trimmed, packed and, optionally, sterilized.

Sterilization can in particular take place by radiation with gamma rays and/or with electron rays after the solvent dehydration. The preservation and sterilization using solvent dehydration is described in the patent DE 29 06 650. Spongious bone material can alternatively also be processed aseptically such that a separate sterilization is no longer necessary.

CNC milling/drilling machines specific to clean rooms and tools suitable therefor can be used for the processing.

The present invention will be described in the following by way of example with reference to two advantageous embodiments and to the enclosed drawings. There are shown

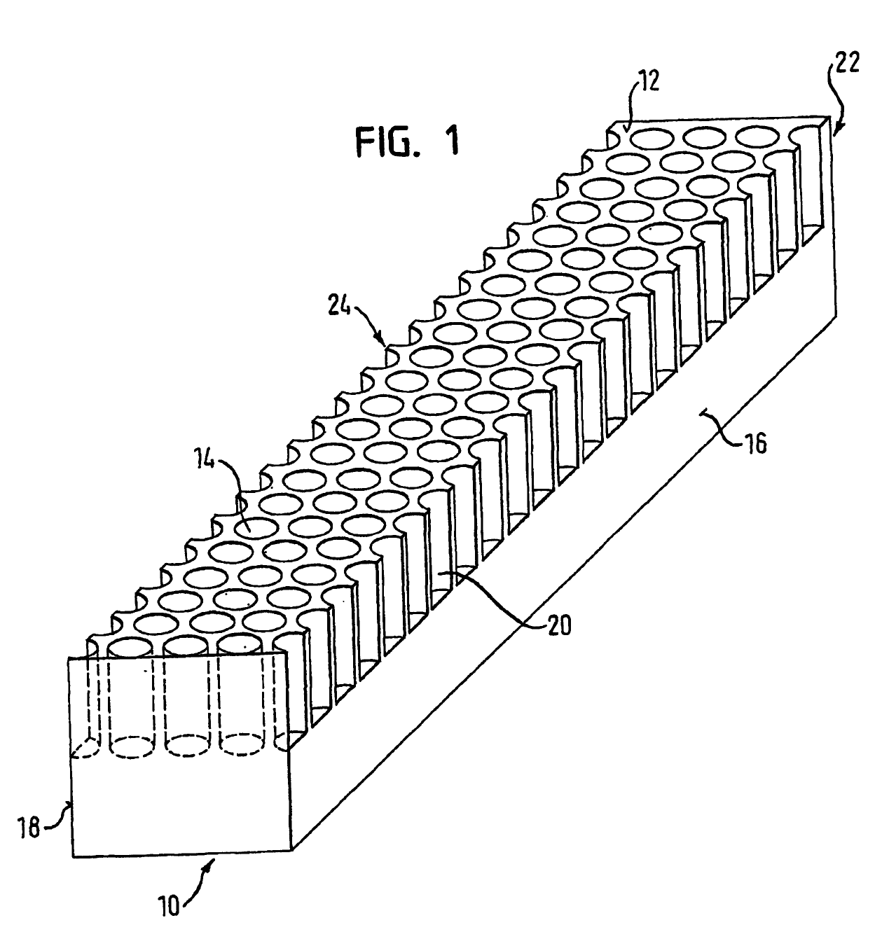

FIG. 1 a schematic, perspective view of a pin-shaped bone implant in accordance with a first preferred embodiment of the invention;

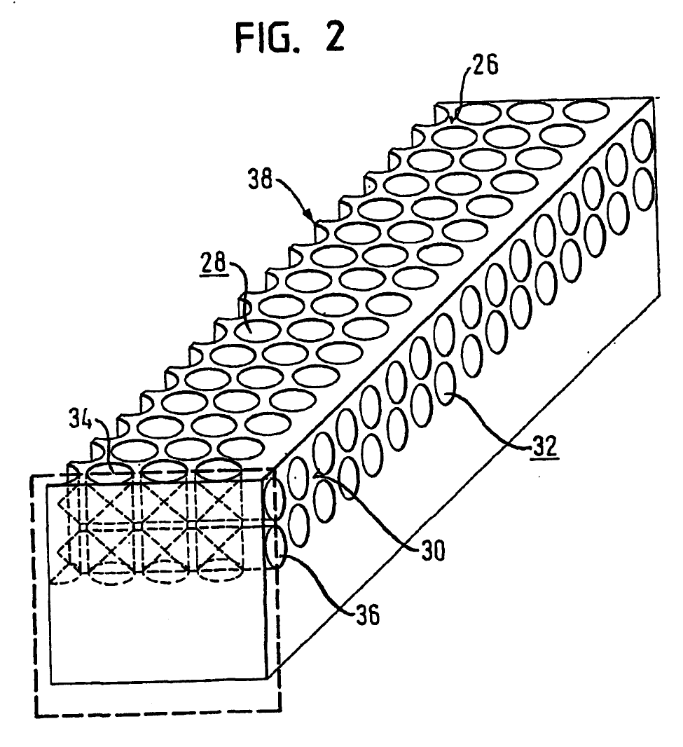

FIG. 2 a schematic, perspective view of a pin-shaped bone implant in accordance with a second preferred embodiment of the invention

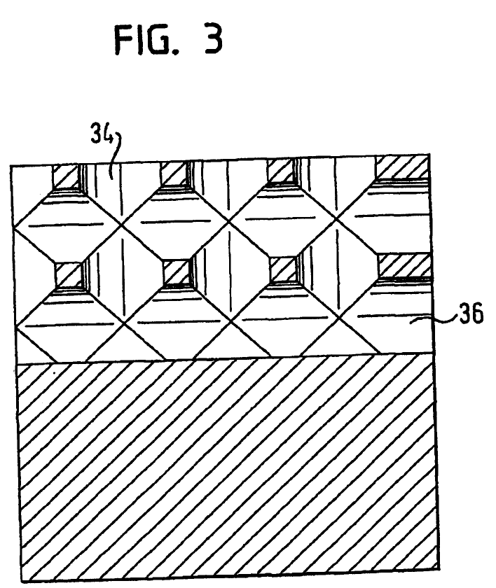

FIG. 3 a sectional view through the bone implant in FIG. 2 along the plane indicated by broken lines in FIG. 2.

In FIG. 1, a pin-shaped bone implant 10 has a surface region 12 with bores 14.

The bone implant 10 has the shape of a right parallelepiped, with the height and the width amounting to approximately 4 mm and the length to approximately 60 mm. The bone implant is made from preserved cortical bone material from cows.

The bores 14 in the surface region 12 of the bone implant 10 are cylindrical and have a diameter or a maximum width of approximately 0.8 mm. The bores are arranged on a square grid and have a spacing of approximately 0.2 mm. This results in a surface density of approximately 100 bores per cm² of the surface 12.

The cylindrical bores 14 are aligned parallel and perpendicular to the surface region 12 and have a depth of approximately 2 mm such that the layer containing the bores has a membrane-like section of a thickness of approximately 2 mm along the surface region 12 with bores 14.

As shown in the Figure, the surface region 12 with bores 14 extends up to the rims of the bone implant. The bores (20) in the edge regions along the side surfaces 16 and 18 open outwardly, whereby in each case a ribbed region 22 or 24 arises with recesses in the form of a half-cylinder. In another embodiment, the ribbing can, in contrast to the Figure, also be peripheral around the surface region 12.

In FIG. 2, a bone implant has, in accordance with a second preferred embodiment, cylindrical bores 28 arranged in a matrix shape extending at regular intervals in a surface region 26 perpendicular to the surface region. In a lateral further surface region 30, further bores 32 are likewise arranged in a matrix shape. Bores 34 of the bores 28 arranged in a row in each case intersect further bores 36 of the further bores 32 arranged in a corresponding column, as shown in FIG. 3, at an angle of approximately 90°. A grid-like system of mutually connected passages is hereby formed in each plane defined by a row and by an associated column such that a number of substantially parallel systems of passages connected in a grid-like manner and corresponding to the number of rows and columns are formed in the implant.

A longitudinal side of the implant in accordance with the second embodiment has a ribbed region 38 which is formed in accordance with the ribbed regions in the first embodiment, but which additionally includes the openings of the further bores 32 not visible in the Figures.

### REFERENCE NUMERAL LIST

10 bone implant
12 surface region
14 bores
16 side surface
18 side surface
20 bore
22 ribbed region
24 ribbed region
26 surface region
28 bores
30 further surface region
32 further bores
34 bores
36 further bores
38 ribbed region

1. A bone implant of spongious and/or cortical bone material of human or animal origin,
in which at least one surface region (12: 26. 30) of the bone implant (10) has a plurality of elongate bores (14; 28, 32) arranged adjacently and not passing through the bone implant characterized in that
the surface region (12; 26) with bores (14; 28) extends up to a edge of the bone implant, with the bores (20) in the edge region being outwardly open and thereby forming a ribbed section (22, 24).

2. A bone implant in accordance with claim 1, characterized in that the bores (14; 28, 34) are arranged in a matrix shape.

3. A bone implant in accordance with claim 1, characterized in that the surface region with the bores (12; 26) forms a membrane section (14; 28).

4. A bone implant in accordance with claim 1, characterized in that the maximum width of the bores (14; 28) lies between approximately 0.05 mm and approximately 2 mm.

5. A bone implant in accordance with claim 1, characterized in that the depth of the bores (14; 28) lies between approximately 0.1 mm and approximately 10 mm.

6. A bone implant in accordance with claim 1, characterized in that the spacing of adjacent bores (14; 28) is not larger than twice their maximum width.

7. A bone implant in accordance with claim 1, characterized in that the surface region (12; 26) has between approximately 20 and 200 bores (14; 28) per cm².

8. A bone implant in accordance with claim 4, characterized in that the spacing of the bores (14; 28) lies between 0.01 mm and the maximum width of the bores.

9. A bone implant in accordance with claim 5, characterized in that the axes of the bores (14; 28) are aligned substantially parallel.

10. A bone implant in accordance with claim 1, characterized in that the bone material between the bores (14) is compact.

11. A bone implant in accordance with claim 1, characterized in that the bone implant has a further surface region (30) into which further bores (32) have been introduced which each at least partly intersect at least one of the bores (28) in the surface region (26).

12. A bone implant in accordance with claim 11, characterized in that at least one of the further bores (32) in the further surface region (30) at least partly intersects at least one of the bores (28) in the surface region (26) at an angle of approximately 90°.

13. A bone implant in accordance with claim 11, characterized in that at least some of the further bores (32) and some of the bores (28) intersecting them in the surface region (26) are formed such that they form a grid of mutually connected bore passages (34, 36).

14. A bone implant in accordance with claim 1, characterized in that the material of the bone implant consists of preserved and sterile bone material.
